(19) United States
(12) Patent Application Publication
(10) Pub. No.: US 2014/0072610 A1
(43) Pub. Date: Mar. 13, 2014

(54) BIOABSORBABLE TRACHEAL STENT, AND METHOD OF MANUFACTURING THEREOF

(71) Applicants: Subramanian Venkatraman, Singapore (SG); Herr Cheun Anthony Ng, Singapore (SG); Yin Chiang Freddy Boey, Singapore (SG); Hsueh Yee Lynne Lim, Singapore (SG)

(72) Inventors: Subramanian Venkatraman, Singapore (SG); Herr Cheun Anthony Ng, Singapore (SG); Yin Chiang Freddy Boey, Singapore (SG); Hsueh Yee Lynne Lim, Singapore (SG)

(73) Assignees: NATIONAL UNIVERSITY OF SINGAPORE, Singapore (SG); NANYANG TECHNOLOGICAL UNIVERSITY, Singapore (SG)

(21) Appl. No.: 14/003,738

(22) PCT Filed: Mar. 21, 2012
(86) PCT No.: PCT/SG2012/000093

§ 371 Date: Nov. 19, 2013

Related U.S. Application Data
(60) Provisional application No. 61/454,858, filed on Mar. 21, 2011.
(60) Provisional application No. 61/465,636, filed on Mar. 22, 2011.

(51) Int. Cl. A61F 2/04; A61K 31/407; A61K 47/32

(52) U.S. Cl. CPC A61F 2/04; A61K 47/32; A61K 31/407
USPC 424/426; 623/9; 514/653; 427/2.25

(57) ABSTRACT

A bioabsorbable tracheal stent is provided. The bioabsorbable stent comprises a biodegradable polymer, wherein the “ biodegradable polymer comprises about 0 to 30 wt % glycerol, polyethylene glycol, triethyl citrate, or mixture thereof. A drug is dispersed within or dissolved in the biodegradable polymer. In a second and third aspect, the invention relates to methods of manufacturing a bioabsorbable tracheal stent. The first method includes forming a solution comprising a biodegradable polymer and a drug, the biodegradable polymer comprising about 0 to 30 wt % glycerol, polyethylene glycol, triethyl citrate, or mixture thereof. The method further comprises casting the solution to form the bioabsorbable tracheal stent. The second method includes forming a polymeric stent, and dip casting the polymeric stent in a solution comprising a biodegradable polymer and a drug to form a coating on the polymeric stent, wherein the biodegradable polymer comprises about 0 to 30 wt % glycerol, polyethylene glycol, triethyl citrate, or mixture thereof.

### CROSS-REFERENCE TO RELATED APPLICATIONS

This application claims the benefit of priority of U.S. Provisional Application Nos. 61/454,858 filed on Mar. 21, 2011, and 61/465,636 filed on Mar. 22, 2011, the contents of which being hereby incorporated by reference in its entirety for all purposes.

### TECHNICAL FIELD

The invention relates to a tracheal stent. In particular, the invention relates to a bioabsorbable tracheal stent.

### BACKGROUND

Trachea airway stenosis results from prolonged endotracheal intubation, tracheotomy, trauma, infections, tumor or tumor-related treatment and congenital disorders. Surgical intervention may be needed to re-establish a patent airway, with insertion of stents to prevent restenosis. Currently available stents include silicone stents, metallic stents, and stents which combine a silicone or synthetic outer coating with metal hoops or mesh.

Silicone stents, such as Dumon®, Montgomery®, and Hood® stents are amongst the most widely clinically used stents. They are well-tolerated, removable and flexible. However, they impair physiologic mucociliary function, trapping airway secretions and mucus plugs, thereby risking life-threatening asphyxia. Silicone stents also have thick walls that narrow the trachea lumen patency, further limiting their use in younger children with small tracheas.

Metallic stents can be inserted endo-tracheally without open surgery, have less trapping of secretions and have thinner walls. However, metallic stents are difficult to remove once they are mucosalized over by epithelium. Furthermore, metallic stents may fragment, extrude and penetrate into neighboring structures, such as the esophagus and large neck vessels, for example.

In view of the above, there is a need for an improved tracheal stent that overcomes at least some of the above drawbacks.

### SUMMARY OF THE INVENTION

In a first aspect, the invention refers to a bioabsorbable tracheal stent. The bioabsorbable stent comprises a biodegradable polymer, wherein the biodegradable polymer comprises about 0 to 30 wt % glycerol, polyethylene glycol, triethyl citrate, or mixture thereof. A drug is dispersed within or dissolved in the biodegradable polymer.

In a second aspect, the invention refers to a method of manufacturing a bioabsorbable tracheal stent. The method comprises forming a solution comprising a biodegradable polymer and a drug, the biodegradable polymer comprising about 0 to 30 wt % glycerol, polyethylene glycol, triethyl citrate, or mixture thereof. The method further comprises casting the solution to form the bioabsorbable tracheal stent.

In a third aspect, the invention refers to a method of manufacturing a bioabsorbable tracheal stent. The method comprises forming a polymeric stent, and dip casting the polymeric stent in a solution comprising a biodegradable polymer and a drug to form a coating on the polymeric stent, wherein the biodegradable polymer comprises about 0 to 30 wt % glycerol, polyethylene glycol, triethyl citrate, or mixture thereof.

In a fourth aspect, the invention refers to a bioabsorbable tracheal stent formed by a method according to the second aspect or the third aspect.

### BRIEF DESCRIPTION OF THE DRAWINGS

The invention will be better understood with reference to the detailed description when considered in conjunction with the non-limiting examples and the accompanying drawings, in which:

FIG. 1 is a series of photographs showing A) a helical-shaped stent; B) a tubular-shaped stent, and C) a tubular-shaped stent with 0.1 mg mitomycin C (MMC).

FIG. 2 is a graph depicting the release profile of 0.1 mg MMC for a duration of 12 weeks (84 days).

FIG. 3 is a photograph showing endoscopic findings in Control Group 1 with diathermy injury to trachea without stenting 6 weeks after diathermy.

FIG. 4 is a photograph showing endoscopic findings in Control Group 2—Commercial Silicone Tubular-shaped Stent at 4 weeks after stent implantation, show mucus trapping throughout the silicone stent narrowing the tracheal airway.

FIG. 5 is a photograph showing endoscopic findings in Group 3—Bioabsorbable Helical-shaped Stent at 6 weeks after stent implantation. Severe granulation tissue formation and significant mucus trapping was noted between the helical stent coils (non-stented trachea areas).

FIG. 6 is a photograph showing endoscopic findings in Group 4—Bioabsorbable Tubular-shaped Stent at 6 weeks after stent implantation. The mucus trapping and trachea narrowing was similar to Control Group 2—Commercial Silicone Tubular-shaped Stent, but less than Group 3—Bioabsorbable Helical-shaped Stent.

FIG. 7 is a photograph showing endoscopic findings in Group 5—Bioabsorbable Tubular-shaped Stent with MMC at 6 weeks after stent implantation. There was less granulations compared to Silicone and Bioabsorbable Tubular-shaped without MMC stents.

FIG. 8 is a graph depicting extent of tracheal stenosis in all 5 groups. Group 5—Bioabsorbable Tubular-shaped Stent with MMC had the least trachea stenosis from granulations and mucus plugging. Data points with (*) indicate that only 1 surviving rabbit in that group from that week onwards was used for stenosis grading.

FIG. 9 is a table (Table 1) summarizing the number of unscheduled rabbit deaths/euthanasia in all groups (Control Groups 1 and 2; and Groups 3 to 5).

FIG. 10 is a series of photographs of a bioabsorbable tracheal stent according to various embodiments of the invention. (A) The embodiment shown is a tubular bioabsorbable tracheal stent having rectangular holes distributed in the body of the stent. (B) The embodiment shown is a tubular bioabsorbable tracheal stent having diamond shaped holes distributed in the body of the stent.

FIG. 11 is a photograph of a bioabsorbable tracheal stent according to another embodiment of the invention. The embodiment shown is a tubular bioabsorbable tracheal stent having rectangular holes distributed in the body of the stent. Due to the incorporation of MMC into the body of the stent, the stent shown in the photograph is red, or darker in color compared to the embodiment shown in FIG. 10(A).

FIG. 12 is a graph depicting the degradation profile of a stent material, plotted in terms of weighted-average molecular weight (MW) versus time (weeks).

FIG. 13 is a graph depicting the degradation profile of a stent material, plotted in terms of mass loss (%) versus time (weeks).

FIG. 14 is a series of photographs showing (A) a patent trachea (without stent); and (B) a patent trachea (with laser-patterned stent).

### DETAILED DESCRIPTION OF THE INVENTION

In a first aspect, the present invention refers to a bioabsorbable tracheal stent. The terms “bioabsorbable”, “biodegradable” and “bioresorbable” are used interchangeably herein, and refers to the ability of a material to degrade or breakdown over a period of time due to the chemical and/or biological action of the body. The term “stent” as used herein refers to a prosthesis, usually a slotted tube or a helical coil or a wire mesh tube, designed to be inserted into a vessel or passageway of a subject (usually a mammal such a human, dog, mouse, rat, etc) to be treated to keep it open. A stent of the present invention is designed for use in the trachea or windpipe, and may be inserted into a trachea to assist in keeping it open after surgery or to treat a constriction, for example, to allow the passage of air to the lungs.

The bioabsorbable tracheal stent of the present invention comprises a biodegradable polymer. In the context of the present invention, the term “biodegradable polymer” refers to a polymer comprising one or more polymeric components that can be completely removed from a localized area by physiological metabolic processes such as resorption. For example, a biodegradable polymer may, when taken up by a cell, be broken down into smaller, non-polymeric subunits by cellular machinery, such as lysosomes or by hydrolysis that the cells can either reuse or dispose of without significant toxic effect on the cells. Examples of biodegradation processes include enzymatic and non-enzymatic hydrolysis, oxidation and reduction. Suitable conditions for non-enzymatic hydrolysis, for example, include exposure of biodegradable material to water at a temperature and a pH of a lysosome (i.e. the intracellular organelle). The degradation fragments typically induce no or little organ or cell overload or pathological processes caused by such overload or other adverse effects in vivo.

Various examples of biodegradable polymer materials are known in the art, any of which are generally suitable for use as the biodegradable polymer of the present invention. Examples of polymers that are considered to be biodegradable include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, polycarbonates, naturally-occurring biodegradable polymers such as chitosan, collagen, starch, and blends thereof. Examples of polyortho esters include a polylactide, a polyglycolide, a polycaprolactone, a polylactic acid, a biodegradable polyamide, a biodegradable aliphatic polyester, and/or copolymers thereof or with other biodegradable polymers such as those mentioned above.

In various embodiments, the biodegradable polymer is a polymer of an a-hydroxy ester, such as poly(L-lactide), poly(glycolic acid), poly(caprolactone) and copolymers thereof; poly(trimethylene carbonate), poly(hydroxyl butyrate), poly(hydroxyl valerate), poly(dioxanone), and copolymers thereof; biodegradable polyurethanes built with poly(caprolactone)/polylactide soft poly(caprolactone)-trimethylene carbonate soft segments; copolymers thereof, or mixtures thereof.

More specific examples of copolymers which can used in the present invention include copolymers of poly(caprolactone) (PCL) and poly(L-lactide) (PLA). The weight ratio of poly(L-lactide) to poly(caprolactone) in the copolymer may be in the range of about 1:1 to about 9:1, such as about 2:1, about 3:2, or about 7:3. In one embodiment, the biodegradable polymer is a copolymer of poly(L-lactide) and poly(caprolactone) having a weight ratio of about 7:3.

The biodegradable polymer used to form the bio absorbable tracheal stent of the present invention comprises about 0 wt % to 30 wt % glycerol, polyethylene glycol (PEG), triethyl citrate (TEC), or mixtures thereof; such as in the range of about 7.5 wt % to 15 wt %, or about 10 wt %. Glycerol, polyethylene glycol, and triethyl citrate may be used alone or in combination. For example, the biodegradable polymer may comprise about 10 wt % glycerol. As another example, the biodegradable polymer may comprise about 6 wt % polyethylene glycol and about 8 wt % triethyl citrate. As a further example, the biodegradable polymer may comprise about 5 wt % glycerol, about 7 wt % polyethylene glycol, and about 10 wt % triethyl citrate. The glycerol, polyethylene glycol (PEG), and/or triethyl citrate (TEC) may be added to the biodegradable polymer to affect the mechanical properties of the polymer, to render it suitable for the manufacture of a bioabsorbable tracheal stent for trachea stenosis application.

In various embodiments, the biodegradable polymer used to form the bioabsorbable tracheal stent of the present invention comprises glycerol. The glycerol may, for example, be added to increase water uptake into the copolymer, thereby reducing the time required for the biodegradable polymer to degrade. The degradation time of the biodegradable polymer may be reduced to a time period of about 6 weeks to about 3 months, which renders the polymer suitable in manufacture of a bioabsorbable tracheal stent for trachea stenosis applications. In some embodiments, the amount of glycerol used is about 10 wt % of the dry weight of the polymer, which has been found by the inventors of the present invention to be an optimal amount to form the bioabsorbable tracheal stent.

A drug is dispersed within or dissolved in the biodegradable polymer that is used to form the bioabsorbable tracheal stent of the invention. In the context of the invention, the term “drug” generally means a therapeutic or pharmaceutical agent which may be included/mixed into the biodegradable polymer, or impregnated or incorporated into the biodegradable polymer in order to provide a drug-containing stent.

Examples of a drug include, but are not limited to: antiproliferative/antimitotic agents including natural products such as vinca alkaloids (e.g. vinblastine, vincristine, and vinorelbine), paclitaxel, epidipodophyllotoxins (e.g. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiproliferative/antimitotic alkylating agents such as nitrogen mustards (such as mechlorethamine, cyclophosphamide and analogs, melphalan, chlorambucil), ethylenimines and methylmelamines (hexamethylmelamine and thiotepa), alkyl sulfonates-busulfan, nirosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine (cladribine)); platinum coordination complexes (cisplatin, carboplatin), procarbazine, hydroxyurea, mitotane, amino glutethimide; hormones (e.g. estrogen); anticoagulants (heparin, synthetic heparin salts and other inhibitors of thrombin); fibrinolytic agents (such as tissue plasminogen activator, streptokinase and urokinase); antiplatelet (such as aspirin, dipyridamole, ticlopidine, clopidogrel, abciximab); antimigratory; antisecretory (such as breveldin); antiinflammatory: such as adrenocortical steroids (Cortisol, cortisone, fludro cortisone, prednisone, prednisolone, 6-alpha-methylprednisolone, triamcinolone, betamethasone, and dexamethasone), non-steroidal agents (such as salicylic acid derivatives e.g. aspirin); para-aminophenol derivatives (e.g. acetaminophen); indole and indene acetic acids (such as indomethacin, sulindac, and etodalac), heteroaryl acetic acids (such as tolmetin, diclofenac, and ketorolac), arylpropionic acids (such as ibuprofen and derivatives), anthranilic acids (such as mefenamic acid, and meclofenamic acid), enolic acids (such as piroxicam, tenoxicam, phenylbutazone, and oxyphenthatrazone), nabumetone, gold compounds (such as auranofin, aurothioglucose, gold sodium thiomalate); immunosuppressive (such as cyclosporine, tacrolimus (FK-506), sirolimus (rapamycin), azathioprine, mycophenolate mofetil); angiogenic such as vascular endothelial growth factor (VEGF), fibroblast growth factor (FGF); nitric oxide donors; anti-sense oligo nucleotides and combinations thereof.

The drug that is dispersed within or dissolved in the biodegradable polymer may be any therapeutic or pharmaceutical agent suitable for treating cellular proliferation in the trachea. The term “cellular proliferation” as used herein refers to an increase in the number of cells as a result of cell growth and cell division. Through the use of a suitable drug in the bioabsorbable tracheal stent, whereby the drug may be introduced onto the mucosa immediately following dilatation of the trachea due to insertion of the tracheal stent, the incidence of re-stenosis is decreased by decreasing the production of fibroblasts and scar tissue. Specific examples of drug that may be used include mitomycin C (MMC), dexamethasone (DXM), and/or fluracil (5-FU). In one embodiment, the drug is mitomycin C.

The drug may be dispersed within or dissolved in the biodegradable polymer. For example, the drug may be present as particles within a polymeric matrix formed from the biodegradable polymer. In other embodiments, the drug may first be dissolved in the polymeric blend, prior to use of the polymeric blend to form the bioabsorbable tracheal stent. In various embodiments, the drug is homogeneously dispersed within or dissolved in the biodegradable polymer, such that drug elution from the stent is at least substantially uniform. The release of the drug from the stent onto the mucosa may also be accomplished by controlled degradation of the biodegradable polymer. After drug elution, the biodegradable polymer should be biodegraded within the body in order to avoid any deleterious effects generally associated with decomposition reactions of polymer compounds in vivo.

The weight percentage of the drug in the bioabsorbable tracheal stent may be about 0 wt % to about 30 wt %, such as about 5 wt % to about 20 wt %, about 10 wt % to about 30 wt %, or about 20 wt % to about 30 wt %. In various embodiments, the bioabsorbable tracheal stent consists essentially of a biodegradable polymer, the biodegradable polymer comprising abou 0 to 30 wt % glyeerol, polyethylene glycol, triethyl citrateor mixture thereof, and a drug dispersed within or dissolved in the biodegradable polymer. In other embodiments, the biodegradable tracheal stent consists of the biodegradable polymer, the biodegradable polymer comprising about 0 to 30 wt % glycerol, polyethylene glycol, triethyl citrate, or mixture thereof; and a drug dispersed within or dissolved in the biodegradable polymer.

The biodegradable polymer comprising glycerol, polyethylene glycol, triethyl citrate, or mixture thereof, and a drug dispersed within or dissolved therein may form the body of the bioabsorbable tracheal stent. The body of the stent may be helicoidal or tubular. In various embodiments, the bioabsorbable tracheal stent is tubular in shape. For example, the bioabsorbable tracheal stent may have a hollow cylindrical configuration. The stent may have any suitable size defined in terms of length, outer diameter and wall thickness, for example, for application as a tracheal stent.

In various embodiments, the bioabsorbable tracheal stent may be designed to fit a pediatric tracheal airway. In these embodiments, the bioabsorbable tracheal stent may have a length of about 8 mm to about 12 mm, such as about 8 mm, 9 mm, 10 mm, 11 mm, or about 12 mm. In one specific embodiment, the length of the bioabsorbable tracheal stent is about 10 mm. The outer diameter of the bioabsorbable tracheal stent may be in the range of about 5 mm to about 6 mm, such as about 5 mm, 6 mm or 7 mm. In various embodiments, the outer diameter of the bioabsorbable tracheal stent is about 6 mm. The thickness of the wall of the bioabsorbable tracheal stent may range from about 0.2 mm to about 1 mm, such as about 0.2 mm to about 0.5 mm, about 0.2 mm to about 0.3 mm, or about 0.25 mm.

In embodiments in which the bioabsorbable tracheal stent is tubular in shape, the tubular bioabsorbable tracheal stent may comprise holes distributed throughout the stent. The holes may be of any suitable shape, such as rectangular, diamond, circle or ellipsoidal, or irregularly shaped. In some embodiments, the holes are rectangular. The holes may be of any suitable size, and/or comprise a range of sizes and shapes. The holes may be formed using any suitable method, such as laser cutting, mechanical cutting, and chemical etching. The holes may be distributed evenly throughout the stent to allow preservation of mucosa within the stented area of trachea.

In alternative embodiments, the biodegradable polymer, comprising about 0 to 30 wt % glycerol, polyethylene glycol, triethyl citrate, or mixture thereof, and a drug dispersed within or dissolved therein, forms a coating on a polymeric stent. The polymeric stent may comprise a biodegradable polymer that is the same as or different from the biodegradable polymer of the coating. In some embodiments, the polymeric stent comprises a biodegradable polymer that is the same as the biodegradable polymer of the coating. In some embodiments, the polymeric stent comprises a biodegradable polymer that is different from the biodegradable polymer of the coating. In various embodiments, the polymeric stent is formed entirely from a biodegradable polymer that is different from the biodegradable polymer of the coating. The choice of polymer to use for the coating and the polymeric stent may depend on a number of factors, such as the degradation time required for the stent and the type of drug that is comprised in the coating and/or polymeric stent.

The biodegradable polymer of the polymeric stent may further contain a drug, which may be the same as or different from the drug comprised in the coating. For example, a different drug may be used in the coating and in the polymeric stent for a more tailored treatment procedure, whereby a drug comprised in the coating may first be dispensed to the patient when the coating degrades, while a drug comprised in the polymeric stent may be dispensed at a later stage upon subsequent degradation of the stent.

In embodiments in which the same drug is comprised in the polymeric stent and in the coating, the drug may be present in a different concentration in the polymeric stent and in the coating, which may be customized to the specific requirements of the intended application of the bioabsorbable tracheal stent. For example, a higher concentration of the drug may be present in the coating for a more aggressive post-surgery treatment during the initial stages, while a lower concentration of the drug may be used in the polymeric stent for a milder treatment at later stages. In various embodiments, the drug may also be present in different forms in the coating and in the polymeric stent. For example, the drug that is present in the polymeric stent may be in the form of particles dispersed therein, whereas the drug that is present in the coating may be at least substantially dissolved therein.

In various embodiments where mitomycin C is used as the drug, the bioabsorbable trachea stent of the invention is able to achieve sustained mitomycin C drug elution for preventing trachea stenosis.

The bioabsorbable tracheal stent according to various embodiments of the invention are advantageous over conventional non-bioabsorbable tracheal stents, as they provide temporary rigidity before bioabsorption time-frame, and do not need removal during another general anesthesia. Furthermore, bioabsorbable stents can be thin-walled compared to silicone stents for example, and allow sustained drug,elution to prevent restenosis.

In a second aspect, the invention relates to a method of manufacturing a bioabsorbable tracheal stent. The method comprises forming a solution comprising a biodegradable polymer and a drug, the biodegradable polymer comprising about 0 to 30 wt % glycerol, polyethylene glycol, triethyl citrate, or mixture thereof. In some embodiments, the biodegradable polymer comprises about 10 wt % glycerol. Examples of biodegradable polymer and drug that may be used have already been described herein.

The term “solution” as used herein generally refers to a liquid having a substance dissolved in the liquid. The term is also used to refer to a liquid having a substance dispersed therein. For example, the solution comprising a biodegradable polymer and a drug may be formed by adding the biodegradable polymer to a suitable solvent, with subsequent addition of the drug. Generally, the order in which the biodegradable polymer or the drug is added to the solvent is inconsequential, i.e. either the biodegradable polymer or the drug may be added to the solvent first, or they may be added at the same time.

Either one of or both the biodegradable polymer and the drug may be dissolved in the solvent. The choice of solvent to be used may depend on the biodegradable polymer that is used. Examples of solvent that may be used include, but are not limited to, water, organic solvents such as hydrocarbons (e.g. pentane, hexane, cyclohexane, etc.), ethers (diethylether, tetrahydrofurane, dioxane, etc.), esters including diethlyester etc, halogenated organic solvents such as chloroform, dichloromethane, diehloroethane, etc., or aromatic hydrocarbons (e.g. benzene, toluene, etc.).

In other embodiments, the biodegradable polymer may be in the form of a liquid, for example, a liquid polymer blend. In these cases, a solvent may not be required to form the solution, and the drug may be added directly to the biodegradable polymer.

The method of manufacturing a bioabsorbable tracheal stent according to the invention includes casting the solution to form the bioabsorbable tracheal stent. The term “casting” as used herein refers to forming a layer of a material by depositing on a surface, a solution comprising the material, and removing the solvent or liquid comprised in the solution. For example, the solution may be cast on a suitable mold to form a thin film, wherein the resultant thin film may assume the shape of the bioabsorbable tracheal stent.

In various embodiments, the bioabsorbable tracheal stent is tubular in shape. To form a tubular bioabsorbable tracheal stent, the solution may, for example, be cast on a rod-shaped mold to form a thin film around the mold, with subsequent drying of the solution and removal of the mold to form the tubular bioabsorbable tracheal stent.

In various embodiments, removal of the solvent or liquid comprised in the solution takes place via a drying process. Any suitable drying process, such as oven drying or spray draying, may be used. Drying of the solution may be at any temperature sufficient to drive off the solvent present in the solution. For example, the drying temperature may be in the range from about 25° C. to about 150° C., such as about 25° C. to about 100° C. or about 50° C. to about 150° C.

The method according to the present invention may further comprise forming holes in a tubular stent. For example, the holes may be formed in the tubular stent by laser cutting, mechanical cutting or chemical etching.

In a third aspect, the invention refers to a method of manufacturing a bioabsorbable tracheal stent. The method comprises forming a polymeric stent, and dip casting the polymeric stent in a solution comprising a biodegradable polymer and a drug to form a coating on the polymeric stent, wherein the biodegradable polymer comprises about 0 to 30 wt % of glycerol, polyethylene glycol, triethyl citrate, or mixture thereof. In some embodiments, the biodegradable polymer comprises about 10 wt % glycerol. Suitable materials to form the polymeric stent may include a biodegradable polymer, which may be the same as or different from the biodegradable polymer comprised in the solution. Examples of biodegradable polymer and drug that may be used have already been described herein.

The polymeric stent may be formed by any known methods, such as, but not limited to, molding, extrusion and laser cutting. In embodiments in which the polymeric stent is formed by molding, a pre-polymer solution may first be introduced into a mold, and subsequently cured or hardened using ultraviolet radiation, electron beam, heat or chemical additives, for example, to form the polymeric stent. In embodiments in which the polymeric stent is formed by extrusion, a polymer melt may be conveyed through an extruder which is then formed into a tube. In embodiments in which the polymer stent is formed by laser cutting, a laser such as a UV laser, excimer laser or other known lasers may be used to cut a sheet of polymer or a polymer tube to form the polymeric stent. In further embodiments, patterns may be cut into the polymeric stent using laser cutting.

The method of the present invention includes dip casting the polymeric stent in a solution comprising a biodegradable polymer and a drug to form a coating on the polymeric stent, wherein the biodegradable polymer comprises about 0 to 30 wt % of glycerol, polyethylene glycol, triethyl citrate, or mixture thereof. In some embodiments, the biodegradable polymer comprises about 10 wt % glycerol. The term “dip casting” as used herein refers to a process to immerse an object into a liquid or a solution, the liquid or the solution typically comprising a polymer or a pre-polymer, followed by removal of the object, and solidifying the material that is coated on the object into a polymeric material. In various embodiments, the polymeric stent is first coated with a solution comprising a biodegradable polymer and a drug, wherein the biodegradable polymer comprises about 0 to 30 wt % of glycerol, polyethylene glycol, triethyl citrate, or mixture thereof, which is then subjected to a solidification process to harden or solidify the material that is coated on the polymeric stent into a solid coating layer. For example, when the solution comprising a biodegradable polymer and a drug is formed by dissolving or dispersing the polymer and/or the drug in a solvent, the solidification of the solution may take place via drying.

Generally, dip casting of the polymeric stent in the solution may take place at any suitable temperature, such as room temperature, or at a temperature required to maintain the solution comprising a biodegradable polymer and a drug in liquid phase, for example. In various embodiments, dip casting of the polymeric stent in the solution may be repeated for a number of times in order to achieve the required thickness of the coating on the polymeric stent.

In a fourth aspect, the invention refers to a bioabsorbable tracheal stent formed by a method according to the second aspect or the third aspect.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

### EXAMPLE 1

### In-Vitro Mitomycin C (MMC) Release Samples and Analysis

In vitro Mitomycin C (MMC) release studies were performed to simulate MMC release from the drug-loaded tubular stents. Films incorporating MMC were prepared by solution casting. These films (n=3) containing MMC at 0.1 mg/film were immersed in 2 ml of distilled water in glass vials to ensure sink conditions, and placed in a 37° C. incubator, with the medium changed weekly. Drug stability and release were studied using reversed-phase high performance liquid chromatography (HPLC) and measured at a wavelength of 365 nm. After the last time point, extraction of any residual MMC in the films was performed by dissolving all films completely in an organic solvent (tetrahydrofuran) and analyzed by HPLC. Release profiles were normalized based on the total loading determined in this manner.

### EXAMPLE 2

### Stent Fabrication

Two stent designs were used in this study: helical and tubular. Both were fabricated based on the bioabsorbable copolymer, poly(L-lactide-co-ε-caprolactone) (PLLA-PCL) 70/30, from Purac Biochem BV (Gorinchem, The Netherlands). Glycerol, from Sigma-Aldrich Inc. (MO, USA), was added to PLLA-PCL at 10% by weight to increase water uptake into the copolymer, and reduce degradation time to 6 weeks to 3 months for a tracheal stenosis application. MMC was purchased from Hande Industry and Trade Holdings Limited (Shenzhen-China), and its final dosage was optimized to 0.1 mg per-stent.

Sizes of stents chosen to be studied were those that could fit a pediatric tracheal airway. Silicone stents used were tubes with 1 mm wall thickness, 6 mm outer diameter (OD) and 10 mm length. All stents fabricated had 0.25 mm wall thickness, 6 mm+0.2 mm OD and 10 mm length.

Helical-shaped stents were fabricated from PLLA-PCL+10% glycerol strips.

Tubular-shaped stents had 12 rectangular holes cut and distributed throughout each PLLA-PCL +10% glycerol film. For the tubular stent with MMC, MMC was added to the polymer solutions, homogenized and casted.

FIG. 1 is a series of photographs showing A) a helical-shaped stent; B) a tubular-shaped stent, and C) a tubular-shaped stent with 0.1 mg MMC.

### EXAMPLE 3

### Animal Study

All surgical procedures were performed by the same surgeon in an aseptic manner. 5 groups of 5 New Zealand white rabbits in each group, each weighing 3.5 4.0 kg, were studied. Trachea stenosis was created in all groups using unipolar diathermy. The 5 groups were 1) Control 1 —without stent; 2) Control 2—commercially available silicone tubular-shaped stent; 3) Bioabsorbable helical-shaped stent; 4) Bioabsorbable tubular-shaped stent; and 5) Bioabsorbable tubular-shaped stent with MMC.

### EXAMPLE 4

### Surgical Techniques

Each rabbit received ketamine hydrochloride (7.5 mg/kg) and xylazine (10 mg/kg) intramuscularly for general anesthesia and were spontaneously breathing during the 10 minute surgery. The trachea was exposed through a midline vertical skin incision in the neck, strap muscles were retracted laterally and the midline anterior tracheal wall exposed. A midline tracheal incision was made onto the anterior trachea wall between the third and seventh tracheal rings. Unipolar diathermy at 35 watts was used to create mucosa injury and stenosis circumferentially between the 4th to 6th rings. The stents to be studied were than implanted between the 4th and 6th rings. 5-0 nylon suture was used to prevent the stents from dislodging by placing 2 sutures from the stent to the anterior trachea wall.

Each rabbit was observed daily for respiratory distress and well-being. Rabbits with body weight loss of more than 20%, with respiratory distress or anorexia were euthanized. Their airways were evaluated weekly with rigid 2.9 mm diameter 0° endoscopes (Karl Storz Endoscopy, St Louis, Mo.). The endoscopic examinations were digitally recorded. The cross-section and percentage of trachea stenosis were calculated as described by Eliashar (Eliashar et al., 2000, Otolaryngol—Head and Neck Surgery, 122:84-90).

### EXAMPLE 5

### Histology

One rabbit from each group was euthanized every 3 weeks after endoscopic examination. Tracheal tissues were collected immediately after euthanasia and fixed in 10% neutral-buffered formalin for a minimum of 48 hours. Tissues were trimmed, processed routinely for histology and embedded in paraffin. Five micron thick sections were cut and stained with hematoxylin and eosin for morphologic evaluation by light microscopy by a veterinary pathologist.

### EXAMPLE 6

### In Vitro MMC Release Study

In vitro MMC release studies were performed to correlate the in vivo results from implanted tubular stents with MMC. As the implanted stents were subjected to a relatively harsh environment in the rabbits' tracheas with continuous mucus flow and occasional coughing reactions, the in vitro release samples were immersed in water to achieve a release profile mimicking that expected in vivo.

The equation below was used to obtain the kinetic data:

Mₜ/M_{∞}=ktⁿ (1)

where Mₜ/M_{∞} is the fraction or percentage of total drug (M_{∞}) released at time t; k is a constant depending on the conditions of the system; and n is the exponent which describes the diffusional release kinetic mechanism.

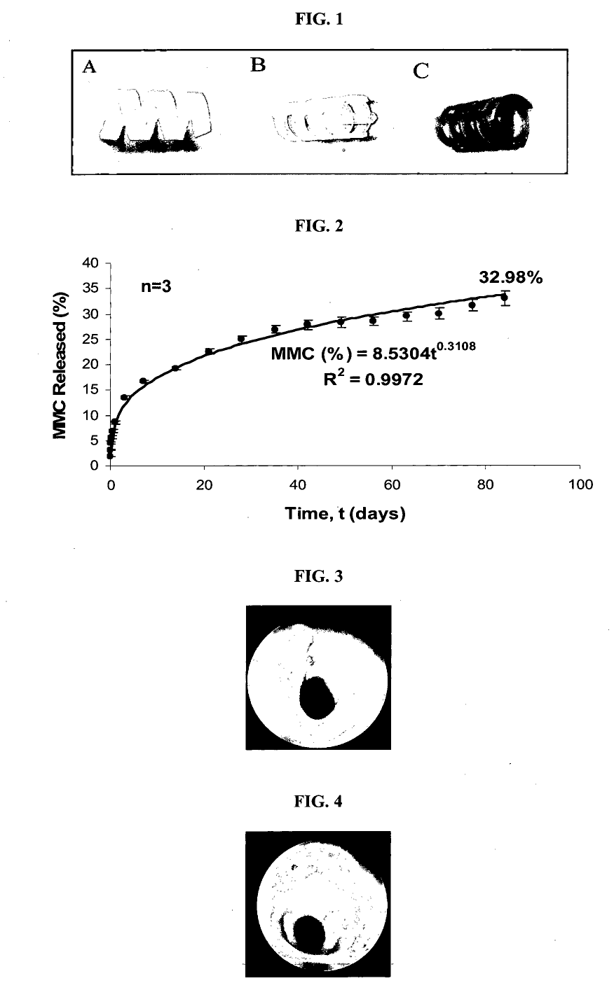

FIG. 2 is a graph depicting the release profile of 0.1 mg MMC for a duration of 12 weeks (84 days). From the results obtained (FIG. 2), a total of only about 33% of the MMC loaded into the bioabsorbable films was released into the media in a 12-week period. The diffusional exponent, n, was 0.3108 and a regression coefficient close to 1 was achieved, indicating the applicability of the equation.

### EXAMPLE 7

### In Vivo Animal Studies

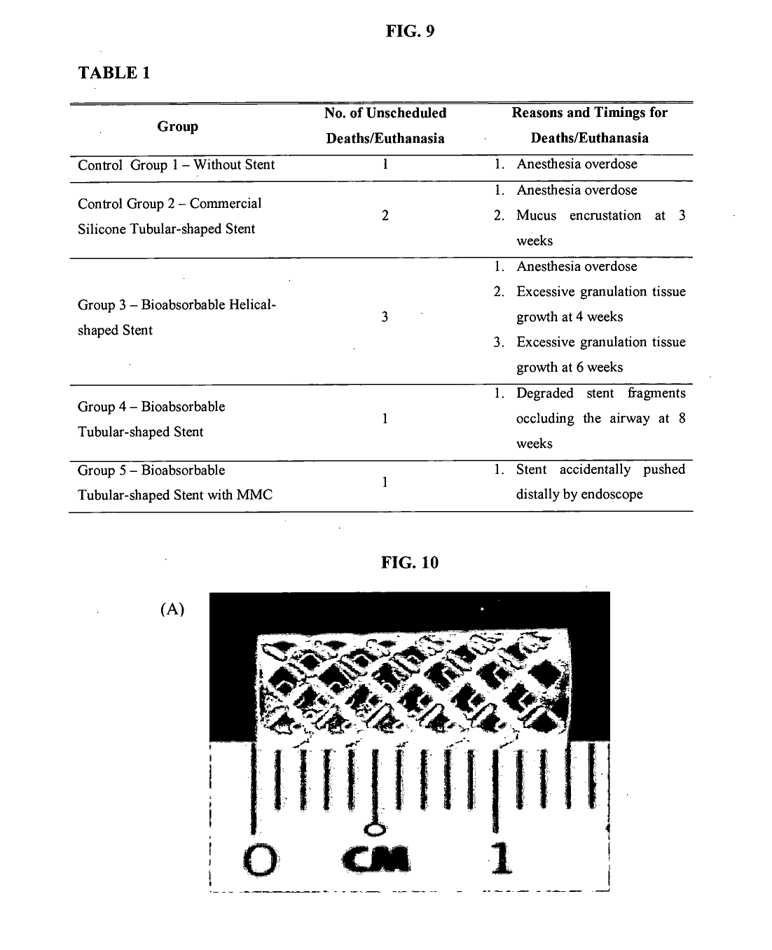

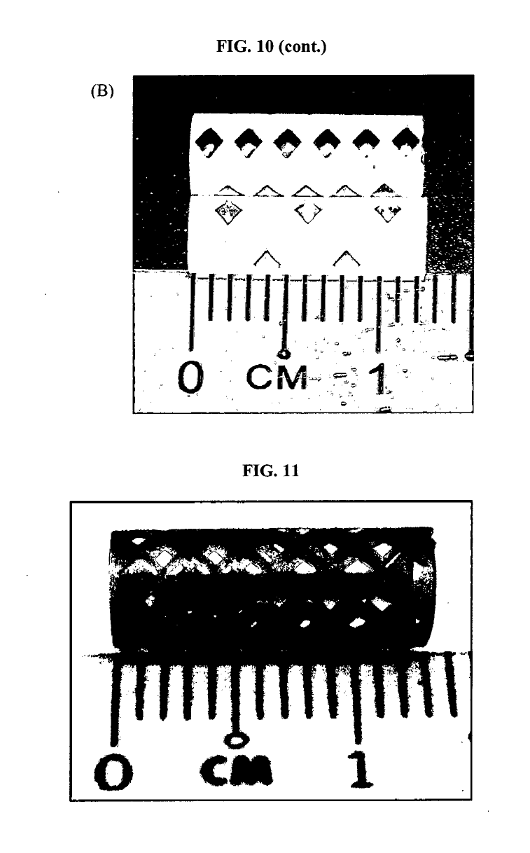

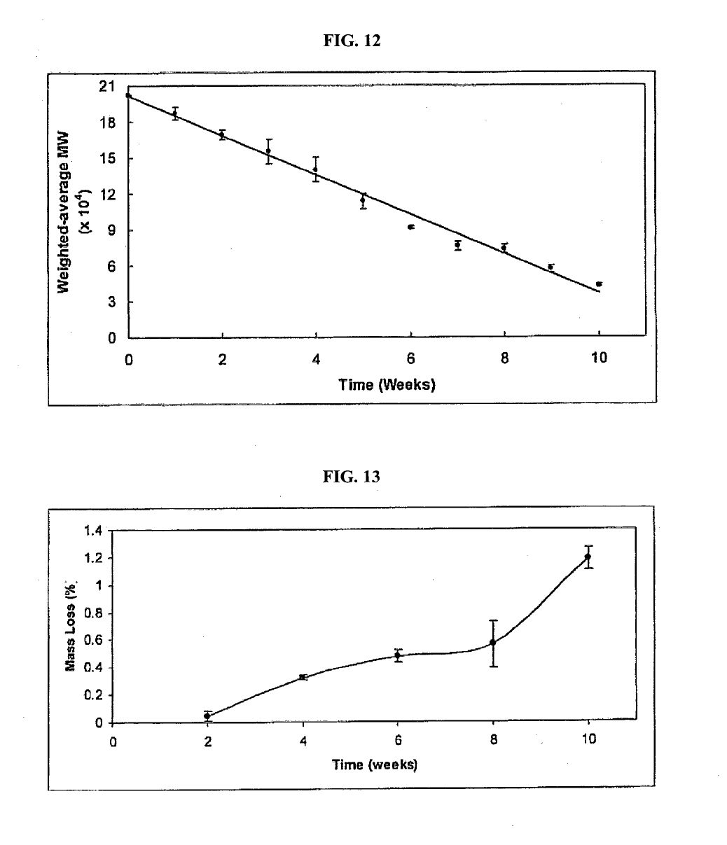

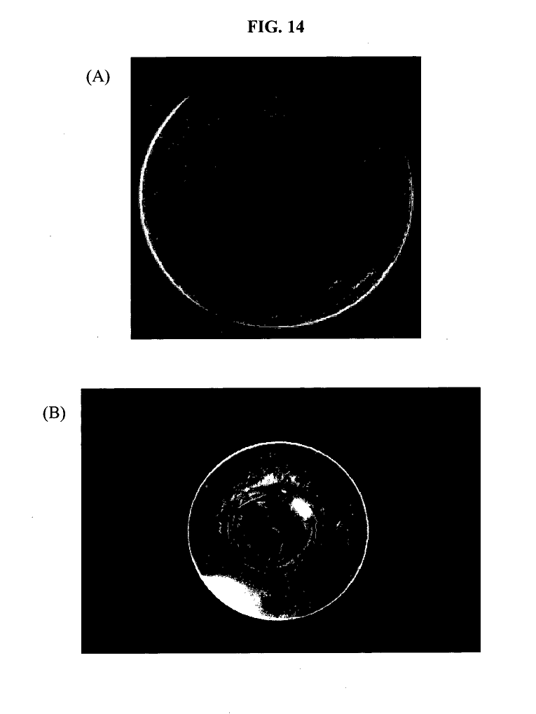

All 25 rabbits recovered well after surgical implantation of the stents. FIG. 9 is a table (Table 1) summarizing the number of unscheduled rabbit deaths/euthanasia in all groups (Control Groups 1 and 2; and Groups 3 to 5).

Referring to the table, five rabbits required euthanasia before their scheduled sacrifice due to respiratory distress (Control Group 2-1; Group 3-2; Group 4-1; Group 5-1). Three rabbits died due to anesthesia drug overdose during endo scopic examinations between week 5 and 7 post insertion of stents (Control Group 1-1; Control Group 2-1; Group 3-1). They were otherwise well before anesthesia, and deaths were not stent related. For subsequent rabbits, only a third of the dosage of anesthesia for implantation of the stents was used during the endoscopies, and earlier reversal from anesthesia was done. There were no further anesthesia-related deaths.

### EXAMPLE 8

### Control Group 1—without Stent

Unipolar diathermy at 35 watts was used to induce stenosis in the trachea of the rabbits. Stable trachea stenosis narrowing the lumen cross sectional area by about 75% was achieved. Stenosis was significant by 3 weeks and stable by 6 weeks post diathermy injury. FIG. 3 is a photograph showing endoscopic findings in Control Group 1 with diathermy injury to trachea without stenting 6 weeks after diathermy.

### EXAMPLE 9

### Control Group 2—Commercial Silicone Tubular-Shaped Stent

Commercially available silicone stents were used to stent the trachea after diathermy injury. One rabbit developed severe post intubation stenosis at the tube cuff site at 3 weeks and this rabbit was euthanized. The remaining 4 rabbits developed cloudy, thick adherent mucus within their stents which resulted in respiratory distress. FIG. 4 is a photograph showing endoscopic findings in Control Group 2 Commercial Silicone Tubular-shaped Stent at 4 weeks after stent implantation, show mucus trapping throughout the silicone stent narrowing the tracheal airway.

### EXAMPLE 10

### Group 3—Bioabsorbable Helical-Shaped Stent

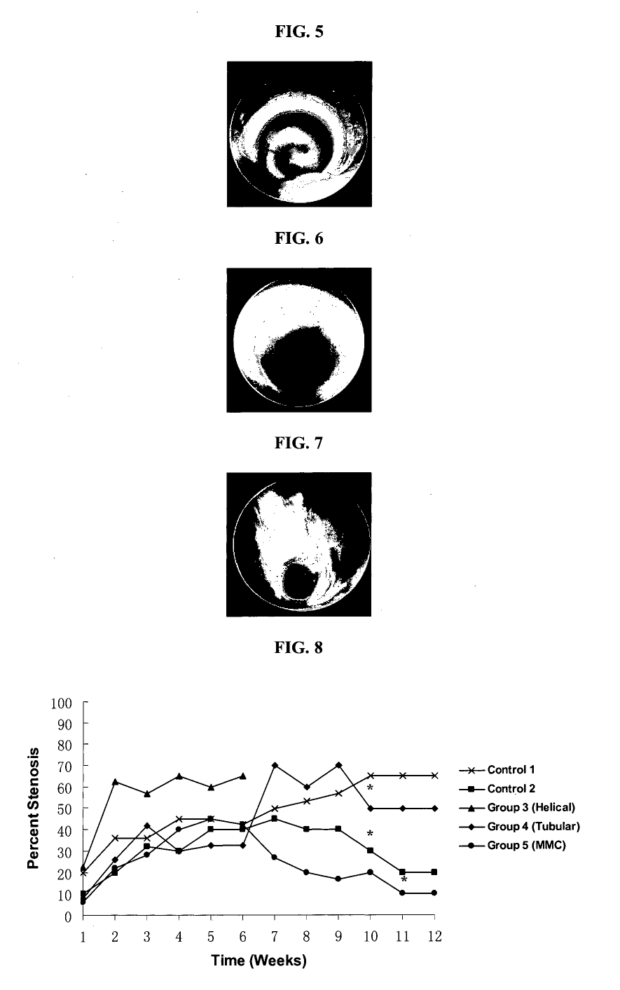

FIG. 5 is a photograph showing endoscopic findings in Group 3 Bioabsorbable Helical-shaped Stent at 6 weeks after stent implantation. Severe granulation tissue formation and significant mucus trapping was noted between the helical stent coils (non-stented trachea areas). The bioabsorbable helical-shaped stents caused profuse tissue reaction in the trachea to develop between the non-stented areas of the trachea between the helices of the stent.

Amongst all the groups, it had the most granulation narrowing and mucus trapping in the trachea lumen.

Two rabbits had to be euthanized at 4 weeks and 6 weeks, and no rabbit survived beyond 6 weeks. In all the other groups, at least 1 rabbit in each group survived up to 12 weeks, and no rabbits had to be euthanized due to excessive granulation tissue growth.

### EXAMPLE 11

### Group 4—Bioabsorbable Tubular-Shaped Stent

The tubular stents unwound to fit the diameters of the tracheal lumens after insertion. The mucus trapping and trachea narrowing due to granulation tissue reaction was similar to Control Group 2—Commercial Silicone Tubular-shaped Stent, but less than Group 3—Bioabsorbable Helical-shaped Stent, evident during the first 3 weeks after stenting.

During the 4th week post stenting, more mucus trapping occurred compared to the silicone stent group. A rabbit in this group died at the 8th week due to obstruction of the trachea by degraded stent fragments. FIG. 6 is a photograph showing endoscopic findings in Group 4—Bioabsorbable Tubular-shaped Stent at 6 weeks after stent implantation.

### EXAMPLE 12

### Group 5—Bioabsorbable Tubular-Shaped Stent with MMC

FIG. 7 is a photograph showing endoscopic findings in Group 5—Bioabsorbable Tubular-shaped Stent with MMC at 6 weeks after stent implantation. There was less granulations compared to Silicone and Bioabsorbable Tubular-shaped without MMC stents. Amongst all groups, this group had the least granulations and mucus trapping. Sustained release of MMC at approximately 200 micrograms/day from these stents showed enhanced efficacy in inhibiting granulation tissue growth. At 11 weeks, 1 stent degraded, split vertically into two parts, and was coughed out by the rabbit. This resulted in granulation and progressive stenosis with blockage of 80% of the tracheal lumen 1 week later.

### EXAMPLE 13

### Extent of Tracheal Lumen Stenosis in all 5 Groups

FIG. 8 is a graph depicting extent of tracheal stenosis in all 5 groups over the follow-up duration of 12 weeks. The tracheal lumen stenosis was most significant in the bioabsorbable helical stents, followed by the group without stent, the bioabsorbable tubular stents and finally the silicone stents. After 12 weeks, trachea stenosis for the bioabsorbable tubular stents with MMC was half that of the silicone stents.

### EXAMPLE 14

### Histology of Tracheas Harvested after Euthanasia

The light microscopic changes seen were similar across all 5 groups and were consistent with injury repair and healing. Mild to moderate submucosal edema, subacute to chronic inflammatory response, granulation tissue formation, and mucosal regeneration were present to some degree in all groups.

### EXAMPLE 15

### Discussion

The rabbit animal model was chosen as its airway diameter is very similar to that for a neonate and young pediatric patient. Furthermore, follow-up endoscopy can be performed in a similar manner to that for human-patients. Trachea stenosis was also created by diathermy heat injury to simulate the conditions of the injured trachea that would benefit from stenting in real life, rather than applying the stents to a normal trachea.

The developed novel bioabsorbable tubular stent with MMC performed the best amongst the bioabsorbable stents. It performed better too than the silicone stent, having the least granulations and mucus trapping and airway obstruction. As the silicone stents are solid tubular stents, granulations will not be found narrowing the stented area of the trachea, only at the proximal and distal ends of the stents. Of the stents tested, the helical stents performed most poorly as the non-stented areas of injured trachea between the helical turns had profuse granulation reactions. For our bioabsorbable tubular stents, we had holes distributed evenly throughout the stent to allow preservation of mucosa even within the stented area of trachea.

Preservation of mucosa is advantageous as healthy mucocilliary activity improves mucus clearance. In future studies, we will study the extent of mucosalization of these bioabsorbable stents. However, as the holes allow some granulations to surface in the stented area of the injured trachea, the bioabsorbable tubular stents with MMC performed the best as MMC inhibits stenosis.

Previous studies of bioabsorbable tracheal stents of various designs in rat or rabbit models involved mainly polymeric materials of poly(L-lactide) (PLLA) and poly(D,L-lactide-co-glycolide) (PLGA). The main problems were excessive granulations, lack of mucosalization over the stent walls, and stent expulsion due to degradation. During in-vitro testing, fragmentation and significant mass loss occurred suddenly for such polymers. To address these problems, we investigated several other bioabsorbable polymer candidates, some with plasticizers added during fabrication of a batch of tracheal stents for an initial feasibility study. Stents fabricated from PLLA-PCL and PLGA with varying amounts of plasticizers; and poly-e-caprolactone (PCL) were implanted in a pilot group of rabbits. PLLA-PCL with 10 wt % of glycerol was the best tolerated material. It maintained its structural integrity throughout the duration of study, and its inherent softness did not induce excessive tissue granulation growth in the trachea. PLLA-PCL+10 wt % glycerol was hence used as the main blend for all bioabsorbable stents fabricated and implanted in this study.

The effect of MMC for inhibition of trachea stenosis remains controversial. Various research groups have reported that restenosis and delayed symptom recurrence were similar after endoscopic dilation with or without MMC. Some other groups have reported that topical MMC effectively prevented scar formation in the aerodigestive tract. In previous studies, MMC could only be applied topically for 1 to 5 minutes to avoid blocking the airway during applications. Though the topical application held 0.1 mg/ml to 1 mg/ml of MMC, the actual amount delivered this way is unknown. This is the first study that investigates MMC application via drug elution from a stent, allowing continuous drug application and a controlled amount of drug delivery.

From the 12-week in-vitro cumulative release profile of films with 0.1 mg MMC (FIG. 2), it appeared that MMC was released via a diffusional release mechanism. The exponent of the diffusion equation is about 0.3, which indicates some deviation from classical Fickian diffusion from a slab geometry (expected n=0.5). Nevertheless, the mechanism is largely diffusion-controlled with about 33% released over 12 weeks. Assuming MMC delivery from the stent to the trachea was uni-directional, a low dosage of 0.0165 mg/stent (0.33×0.1 mg×0.5) would have been sufficient to prevent trachea stenosis if the stent degrades completely in the 12-week period of study. The release of MMC is expected to reach completion when the bioabsorbable polymer begins to degrade significantly at a later stage, changing the release kinetics from diffusion to a more polymer degradation-controlled profile.

In the exemplary experiments carried out, three types of bioabsorbable stents and designs (helical, tubular, and tubular with MMC-elution) were compared to the commercially available silicone tubular stent for the prevention of trachea stenosis. Our novel bioabsorbable tubular stent of PLLA-PCL polymer with Mitomycin C drug elution performed the best, and which is better than the silicone stent. It had the least granulation, mucus trapping and airway obstruction. The results obtained using a biosorbable tracheal stent according to various embodiments of the invention demonstrate that the sustained release of MMC via a bioabsorbable stent in the trachea may prevent trachea stenosis.

1. A bioabsorbable tracheal stent comprising
a) a biodegradable polymer comprising about 0 to 30 wt % glycerol, polyethylene glycol, triethyl citrate, or a mixture thereof; and
b) a drug dispersed within or dissolved in the biodegradable polymer.

2. (canceled)

3. The bioabsorbable tracheal stent according to claim 1, wherein the biodegradable polymer is a copolymer of poly(L-lactide) and poly(caprolactone).

4. The bioabsorbable tracheal stent according to claim 3, wherein the weight ratio of poly(L-lactide) to poly(caprolactone) in the copolymer is about 1:1 to about 9:1.

5. The bioabsorbable tracheal stent according to claim 4, wherein the weight ratio of poly(L-lactide) to poly(caprolactone) in the copolymer is about 7:3.

6. (canceled)

7. The bioabsorbable tracheal stent according to claim 1, wherein the drug is mitomycin C.

8. The bioabsorbable tracheal stent according to claim 1, wherein the biodegradable polymer forms the body of the stent.

9. The bioabsorbable tracheal stent according to claim 1, wherein the biodegradable polymer is a coating on a polymeric stent.

10. The bioabsorbable tracheal stent according to claim 9, wherein the polymeric stent comprises a biodegradable polymer which is different from the biodegradable polymer of the coating.

11. (canceled)

12. The bioabsorbable tracheal stent according to claim 1, wherein the weight percentage of the drug in the bioabsorbable tracheal stent is about 0 wt % to about 30 wt %.

13. The bioabsorbable tracheal stent according to claim 1, wherein the biodegradable polymer comprises about 10 wt % glycerol.

14. The bioabsorbable tracheal stent according to claim 1, wherein the bioabsorbable tracheal stent consists of
a) a biodegradable polymer comprising 0 to 30 wt % of glycerol, polyethylene glycol, triethyl citrate, or a mixture thereof; and
b) a drug dispersed within or dissolved in the biodegradable polymer.

15. (canceled)

16. The bioabsorbable tracheal stent according to claim 1, wherein the tubular bioabsorbable tracheal stent comprises holes distributed throughout the stent.

17. A method of manufacturing a bioabsorbable tracheal stent, comprising
a) forming a solution comprising a biodegradable polymer and a drug, the biodegradable polymer comprising about 0 to 30 wt % glycerol, polyethylene glycol, triethyl citrate, or mixture thereof; and
b) casting the solution to form the bioabsorbable tracheal stent.

18. (canceled)

19. The method according to claim 17, wherein the biodegradable polymer is a copolymer of poly(L-lactide) and poly(caprolactone).

20. The method according to claim 19, wherein the weight ratio of poly(L-lactide) to poly(caprolactone) in the copolymer is about 1:1 to about 9:1.

21. The method according to claim 20, wherein the weight ratio of poly(L-lactide) to poly(caprolactone) in the copolymer is about 7:3.

22. (canceled)

23. The method according to claim 17, wherein the drug is mitomycin C.

24. The method according to claim 17, wherein the biodegradable polymer comprises about 10 wt % glycerol.

25. (canceled)

26. The method according to claim 17, further comprising forming holes in the tubular stent by laser.

27. A method of manufacturing a bioabsorbable tracheal stent, comprising
a) forming a polymeric stent; and
b) dip casting the polymeric stent in a solution comprising a biodegradable polymer and a drug to form a coating on the polymeric stent, wherein the biodegradable polymer comprises about 0 to 30 wt % glycerol, polyethylene glycol, triethyl citrate, or mixture thereof.

28. -30. (canceled)
